(19) United States
(12) Patent Application Publication
(10) Pub. No.: US 2004/0083004 A1
(43) Pub. Date: Apr. 29, 2004

(54) Use of snap-on semiannular augments to inhibit multi-directional instability after total hip arthroplasty

(76) Inventor: Ray C. Wasielewski, New Albany, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 10/612,784

(22) Filed: Jul. 2, 2003

Related U.S. Application Data
(60) Provisional application No. 60/420,565, filed on Oct. 23, 2002.

(51) Int. Cl. A61F 2/32

(52) U.S. Cl. 623/22.24; 623/22.28

(57) ABSTRACT

Particular embodiments of the present invention make use of semiannular stabilizing augments adapted to be mounted about the acetabular cup assembly of the prosthetic hip joint to provide constraint for the joint and to concurrently provide a range of motion desired by patients after surgery, but with the additional benefit of doing so without substantially increasing the risks of dislocation. At least one exemplary embodiment utilizes a semiannular augment formed from biologically reabsorbable material to temporarily constrain the prosthetic ball within the prosthetic acetabular cup. In such an embodiment, it is desired that the biologically reabsorbable material degrades in general proportion to the level of tissue developed by the patient's own body to supplement constraint of the hip joint. Thus, the artificial constraining augments may degrade inversely proportional to the patient's need for inhibition.

### REFERENCE TO RELATED APPLICATION

This application claims the benefit of U.S. Provisional Application Serial No. 60/420,565, entitled “USE OF SNAP-ON SEMIANNULAR AUGMENTS TO INHIBIT MULTI-DIRECTIONAL INSTABILITY AFTER TOTAL HIP ARTHROPLASTY”, filed on Oct. 23, 2002, the disclosure of which is incorporated herein by reference.

### BACKGROUND

1. Field of the Invention

The present invention is directed to insert augments that provide, at least, initial stability in proximity to mammalian joints in response to a surgical procedure. More specifically, the invention is directed toward absorbable and/or insoluble inserts circumferentially positionable in proximity to the hip joint (actual and/or prosthetic) in portions surrounding the acetabular cup, to provide stability to the joint as the patient's tissue develops. Still further, the invention is directed toward biologic and biologically reabsorbable semi-annular inserts that augment joint stability for a hip prosthesis, but which are absorbed over time in proportion to the development of the patient's natural tissue providing more permanent stability.

2. Background of the Invention

Stability after total hip anthroplasty (THA) is one of the most pressing problems in primary and revision acetabular anthroplasty. FIG. 1 is a drawing of a typical prosthesis utilized in THA, which includes an acetabular cup/shell 10 bonded to the pelvis 12 of a patient and a femoral member 14 or stem bonded to a patient's femur 16, where the acetabular cup 10 and femoral member 14 are connected together with a ball-joint coupling 18. If a posterior approach has been done, until the posterior tissues are healed (3-6 months), there is dislocation diathesis in this direction. Similarly, there exists an anterior dislocation tendency after anterolateral approach in the hip. In revision THA cases, periarticular capsular releases for exposure can create multidirectional instabilities.

Conventionally, permanent lips and elevations of varying height and length have been added to the periphery of conventional polyethylene inserts (within the acetabular cup) to augment stability. For example, as shown in a cross-sectional view in FIG. 2, prior art techniques to overcome such instability have utilized permanent constraining rings 20 attached to the periphery of the polyethylene insert 24. However, since the instability is relatively short lived until scar tissue is formed or reformed, permanent elevations and augmented implants such as constraining rings 20 are not always necessary.

These constraining rings 20 may also be the source of impingement and may limit maximal possible range of motion as shown by the angle θ in all 360°. In addition, these constraining rings 20 may not always allow for normal pari-articular scarring to occur to optimize long-term hip stability and range of motion, and may also increase stress transmission to the interfaces of the hip over time resulting in mechanical breakdown of the fixation of the cup to the pelvis or failure of the locking mechanism resulting in backside acetabular wear.

### SUMMARY OF THE INVENTION

The invention is directed toward using insoluble and absorbable materials to provide temporary stability after a surgical joint procedure. Exemplary embodiments of the invention provide biologic and/or biologically reabsorbable insert augments mounted to respective hip prosthesis components. Such biologic and/or biologically reabsorbable augments may be absorbed after a time sufficient for patient tissue formation to provide natural constraint and stability to the joint. As these augments are absorbed, any biologic debris would not be considered third body particulate as, for example, poly-L-lactic acid (PLLA) and porcine small intestinal submucosa (SIS) are not harder than any of the prosthesis components. Likewise, the invention has application in any joint reconstruction where the integrity of the constraining tissue has been compromised by injury or as a result of the surgical procedure itself. Further aspects of the present invention are directed toward using biologic and/or biologically reabsorbable materials loaded with agents that may promote tissue formation, fight infection, and promote clotting. Still further aspects of the present invention are directed to the use of biologically reabsorbable snaps or other fasteners to attach an augment to one of the joint prosthetic components or natural tissue constituents of the joint.

Particular embodiments make use of semiannular stabilizing augments adapted to be mounted about the acetabular cup assembly of the prosthetic hip joint to provide constraint for the joint and to concurrently provide a range of motion desired by patients after surgery, but with the additional benefit of doing so without substantially increasing the risks of dislocation. At least one exemplary embodiment utilizes a semiannular augment formed from biologically reabsorbable material to temporarily constrain the prosthetic ball within the prosthetic acetabular cup. In such an embodiment, it is desired that the biologically reabsorbable material degrades in general proportion to the level of tissue developed by the patient's own body to supplement constraint of the hip joint. Thus, the artificial constraining augments may degrade inversely proportional to the patient's need for inhibition.

It is a first aspect of the present invention to provide a prosthetic device for use with a hip replacement prosthesis that includes an acetabular cup assembly adapted to be fastened to a patient's pelvis and a femoral stem adapted to be fastened to the patient's femur, where the femoral stem includes a ball component at its proximal end received within the acetabular cup assembly to form a ball joint type coupling. The constraining device includes a semiannular augment adapted to be mounted approximate to a rim of an acetabular cup assembly of a hip replacement prosthesis, where the augment assists in improving stability, at least temporarily, of a ball joint type coupling between the acetabular cup assembly and a femoral stem of the hip replacement prosthesis, and, where the semiannular augment is formed from an augment material that is or includes a biologic material, a biologically absorbable material, and/or a combination of biologic and biologically absorbable materials.

It is a second aspect of the present invention to provide a hip prosthesis that includes: (a) an acetabular cup assembly adapted to be fastened to a patient's pelvis; (b) a femoral stem adapted to be fastened to the patient's femur, the femoral stem including a ball component at its proximal end received within the acetabular cup assembly to form a ball joint type coupling; and (c) a semiannular augment mounted to a distal end of the acetabular cup assembly, adjacent to the ball component, where the semiannular augment assists in stabilizing the ball joint type coupling between the acetabular cup assembly and the femoral stem and, where the semiannular augment is formed from an augment material that is or includes a biologic material, a biologically absorbable material, and/or a combination of biologic and biologically absorbable materials.

It is a third aspect of the present invention to provide a prosthetic constraining kit for implantation in proximity to a hip joint that includes a plurality of constraining augments adapted to be individually fastened on an acetabular prosthesis and/or about an acetabular cavity within a hip bone, and that are adapted to be circumferentially positionable about a femoral member (such as a femur and/or a femoral prosthesis), where the constraining augments at least partially define a central aperture allowing the femoral member to extend therethrough, and where the constraining augments allow a range of angular motion of the femoral member while inhibiting a femoral head of the femoral member from completely passing distally through the central aperture.

It is a fourth aspect of the present invention to provide a constraining device for, at least temporarily, promoting engagement of a prosthetic femoral stem component with a prosthetic acetabular component of a prosthetic hip assembly. The constraining device includes a semiannular segment of material that is or includes a biologic material, a biologically absorbable material, and/or a combination of biologic and biologically absorbable materials.

It is a fifth aspect of the present invention to provide a restraining device for, at least temporarily, promoting engagement between a first prosthetic joint component or a first bone component and a second prosthetic joint component or a second bone component. The restraining device includes a restraining material that is a biologic material, a biologically absorbable material, and/or a combination of biologic and biologically absorbable materials, and the restraining device does not circumscribe the first prosthetic joint component, the second prosthetic joint component, the first bone component, and/or the second bone component.

It is a sixth aspect of the present invention to provide a method for providing at least temporary stability to a prosthetic hip joint that includes an acetabular cup assembly bonded to a patient's pelvis and a femoral stem bonded to the patient's femur, where the femoral stem includes a ball component at its proximal end received within the acetabular cup assembly to form a ball joint coupling. The method including the step of mounting a stability enhancement augment to the prosthetic hip joint to improve, at least temporarily, the stability of the prosthetic hip joint, where the stability enhancement augment is formed from an augment material that includes a biologic material, a biologically absorbable material, and a combination of biologic and biologically absorbable materials.

It is a seventh aspect of the present invention to provide a method for providing at least temporary stability to a prosthetic hip joint which includes an acetabular cup assembly bonded to a patient's pelvis and a femoral stem bonded to the patient's femur, where the femoral stem includes a ball component at its proximal end received within the acetabular cup assembly to form a ball joint type coupling. The method including the step of mounting a plurality of individual constraining augments on an acetabular prosthesis, where the individual constraining augments at least partially define a central aperture for allowing a femoral component to extend therethrough while inhibiting a femoral head of the femoral component from completely passing distally through the central aperture.

### BRIEF DESCRIPTIONS OF THE DRAWINGS

FIG. 1 is a perspective view, from below, of a prosthetic hip assembly for use in total hip arthroplasty;

FIG. 2 is a cross-sectional schematic view of a prosthetic hip assembly incorporating a permanent constraining ring;

FIG. 3 is an overhead view of an acetabular cup assembly incorporating an exemplary embodiment of the present invention;

FIG. 4 is a cross-sectional, exploded view of a prosthetic hip assembly incorporating an exemplary embodiment of the present invention;

FIG. 5 is a cross-sectional view of a prosthetic acetabular component incorporating another exemplary embodiment of the present invention;

FIG. 6 is an overhead view of a prosthetic acetabular cup component incorporating an additional exemplary embodiment of the present invention;

FIG. 7 is a cross-sectional, exploded view of a prosthetic acetabular cup assembly incorporating an additional exemplary embodiment of the present invention;

FIG. 8 is a cross sectional view of a plurality of exemplary embodiments of the present invention adapted to be mounted to a prosthetic hip assembly; and

FIG. 9 is an overhead view of a prosthetic acetabular cup component incorporating another exemplary embodiment of the present invention.

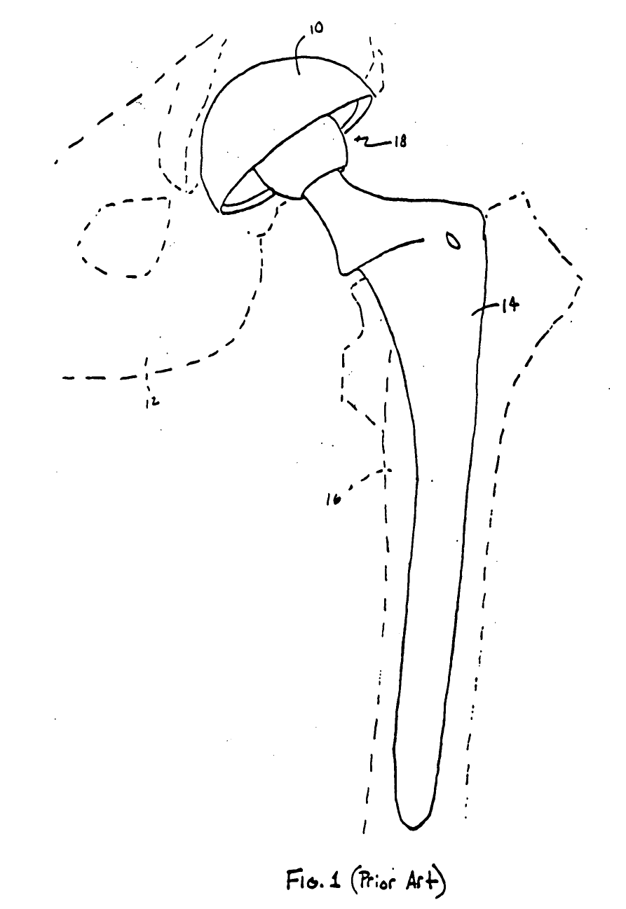

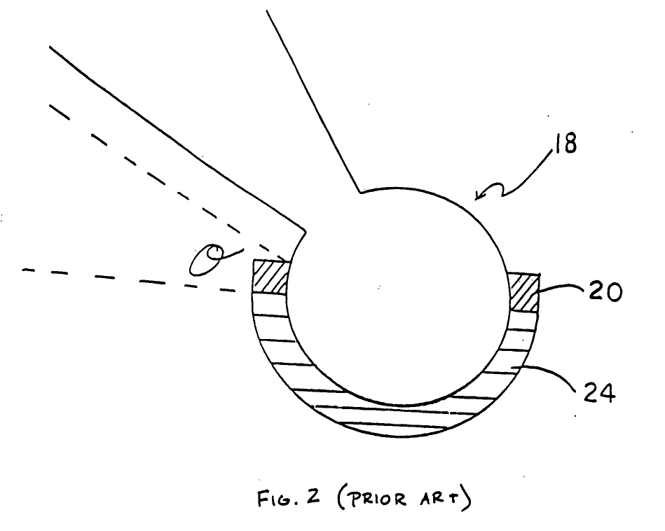

### DESCRIPTION OF THE PRESENT INVENTION

The exemplary embodiments described herein relate to stabilizing devices and techniques for use in joint reconstruction and replacement surgery. The inventions described herein will be described with respect to hip reconstruction and total hip arthroplasty, however, it should be understood that these exemplary applications do not limit the scope of the invention to such applications. Thus, it will become apparent to those of ordinary skill in the art that the devices and techniques disclosed herein may be useful for other types of implants and orthopedic surgeries.

For the purposes of this disclosure, “biologic material” refers to material being derived or synthesized from living organisms, cell, tissues, and/or their products. A biologic material may or may not be a biologically reabsorbable material.

For the purposes of this disclosure, “biologically reabsorbable material” refers to material that is biologically compatible with the mammalian body having the capacity to degrade within, dissolve within, and/or be absorbed by the mammalian body. A biologically reabsorbable material may be a biologic material, a naturally occurring material, or a synthetic material.

For the purposes of this disclosure, “biologically acceptable material” refers to material that is biologically compatible with the mammalian body that may not necessarily degrade within, dissolve within, and/or be absorbed by the mammalian body. A biologically acceptable material may be, for example, a biologic material, a biologically reabsorbable material, a naturally occurring material or a synthetic material.

For the purposes of this disclosure, the numeral “26” will be used to refer generally to all embodiments of the augments discussed herein. Specific embodiments will be referred to as “26a”, “26b”, etc.

The invention, in an exemplary embodiment, provides biologic or biologically reabsorable insert augments that provide initial stability in selected circumferential portions of the acetabular cup, but which may be absorbed over time as the patient's muscles and ligaments heal. Insert augments comprising biologically acceptable material may also be utilized in place of, or in combination with the biologic or biologically reabsorbable augments if desired. The augments may be placed anteriorly with respect to the acetabular cup assembly, for example, to mitigate anterior dislocation when the hip is externally rotated and extended. These augments may also or alternatively be located posteriorly to mitigate posterior dislocation when the hip is flexed and internally rotated. Such augments sometimes characterized herein as semiannular augments, may snap into place along desired circumferential portions of the rim of the acetabular cup assembly and allow multiple semiannular augments to be utilized in cases of multidirectional instability. Additionally, areas may be left unaugmented along the rim to reduce femoral neck impingement with the acetabular cup assembly with certain angular movements of the femoral stem, and to, in turn, optimize immediate postop range of movement (ROM) and stability. Advantageously, the augments may be mounted to the rims of non-polyethylene hard-bearing inserts of the acetablular cup assembly that often cannot utilize conventional elevations and lips due to impingement and subsequent accelerated wear concerns (from third body particulate debris).

Certain aspects of the invention are directed to a system of modular, “snap-on” augments that provide initial stability (constrainment and/or restrainment) along the rim of the acetabular cup and/or insert bearing in the locations desired. In certain detailed aspects of the invention, such semiannular augments may be biologic, biologically reabsorbable, permanent, and/or combinations of such.

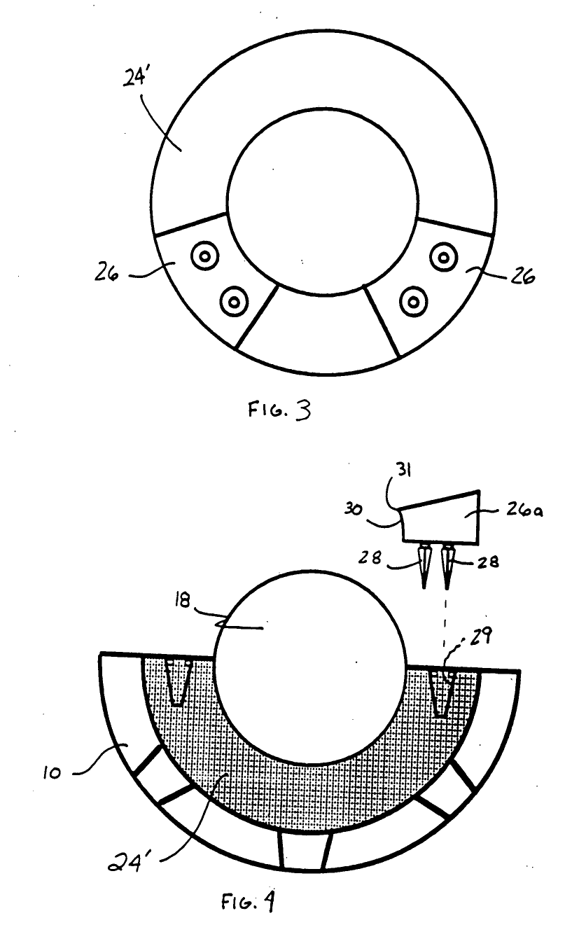

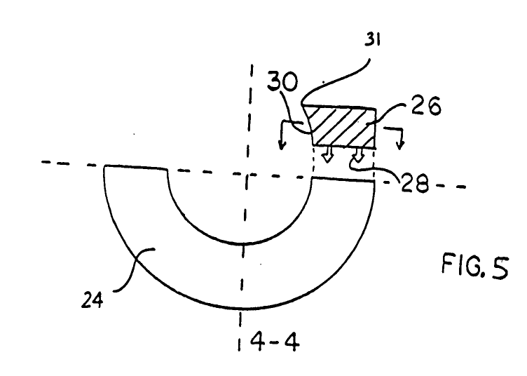

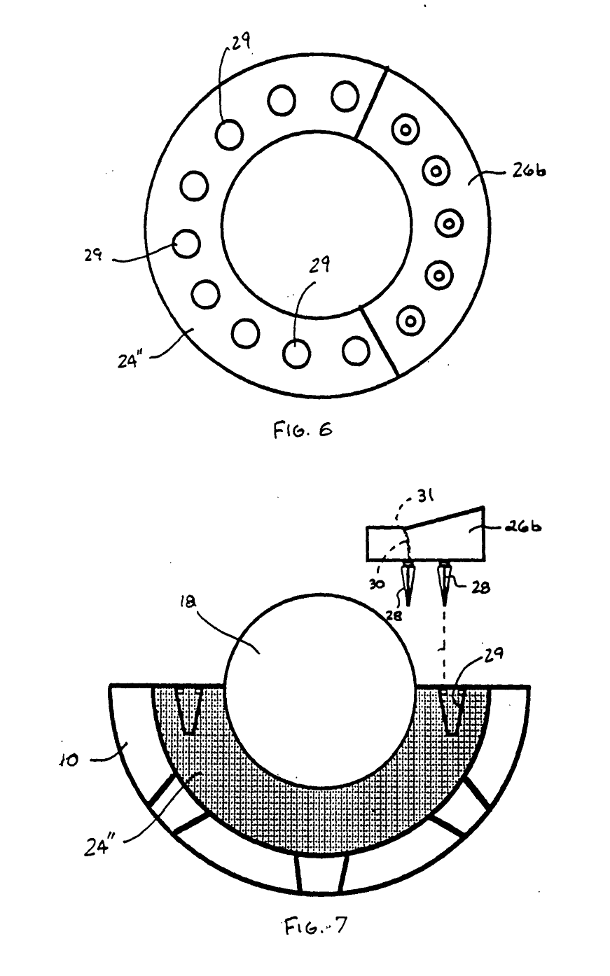

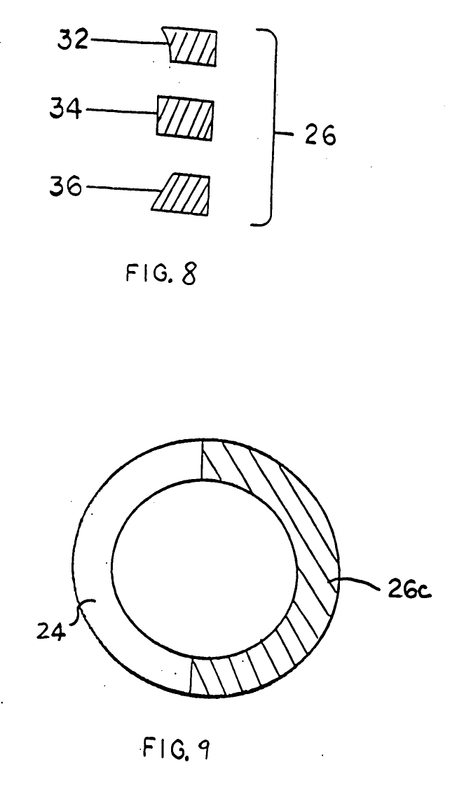

As shown in FIGS. 3 and 4, a bearing insert 24′ according to an exemplary embodiment of the present invention includes a pair of semiannular augments 26a attached thereto along certain circumferential regions of the distal face of its rim. FIG. 4 provides a cross-sectional, exploded, side-view of the exemplary embodiment shown in FIG. 3, which also displays the ball component 18 of the femoral member and the bearing insert 24′ seated within the acetabular cup 10.

Referring specifically to FIG. 4, the snap-on semiannular augments 26a according to the present embodiment may be attached at two places circumferentially around the rim of the insert bearing 24′ using male couplings 28 that are received within and coupled to female couplings 29 provided in the distal face of the insert bearing 24′ rim. The male couplings 28 may be snap-on mechanisms such as resilient cones (as illustrated) that collapse when being inserted into a narrowed opening (annular shoulder) in the female indentation 29 and expand again when passing the shoulder to be retained within the indentation 29 by the shoulder. Screw mechanisms, snaps, clips, keyways, dowels, adhesives and rivets may also be utilized.

The snap-on semiannular augments 26 in this exemplary embodiment include a semi-spherical shaped inner-radial side surface 30 to provide a radially-inwardly extending lip 31 that acts to constrain the ball component 18 of the femoral member within the acetabular cup assembly.

As shown in FIG. 5, axis 4-4 shows the range of movement/constrainment available to the femoral component 14. In the upper right quadrant, the femoral ball is constrained by the augment 26 to help maintain the ball-joint coupling with the acetabular cup 10, but range of angular motion of the femoral stem to this quadrant is simultaneously limited by this augment 26. Conversely, in the upper-left quadrant, the femoral ball is not constrained, and in turn, the range of angular movement of the femoral stem to this quadrant is not as limited. Therefore, the total range of movement available to the patient's femoral component 14 need not be impaired in all 360° about the rim of the acetabular cup assembly to maintain the ball-joint coupling 18 within the acetabular cup/shell 10.

As shown in FIGS. 6 and 7, the snap-on semiannular augment 26b according to this additional embodiment may be attached various places circumferentially around the insert bearing 24″ using male couplings 28 that are received within and coupled to female couplings 29 provided in the distal face of the insert bearing 24″. In this embodiment, the female couplings 29 are distributed uniformly about the distal face of the bearing to allow the augment 26b to be selectively place at various desired positions.

FIG. 8 illustrates exemplary shapes (32, 34, 36) of the snap-on semiannular augments 26 in accordance with the present invention. Shape 32 is primarily for constraining purposes, while shapes 34 and 36 are primarily for restraining purposes.

The snap-on semiannular augments 26 can have several different arcuate lengths to choose from (compare FIGS. 3, 5 & 9, for example, and without limitation); and can also have several cross-sectional profiles of varying shapes and dimensions to choose from (see FIG. 8, for example, and without limitation) depending upon the type and extent of constraint/restraint desired.

FIG. 9 illustrates an alternative exemplary embodiment of the biologically absorbable snap-on augments 26c having an arcuate length approximately half of the circumference of the acetabular insert 24. It will also be appreciated that not all augments need to be semiannular in shape to fall within the scope of certain aspects of the present invention.

The snap-on semiannular augments 26 of the invention may be formed from a biologically acceptable material that inhibits corrosion and attack from the patient's body while continuing to provide stability to the hip joint. Beneficially, these snap-on augments 26 and snap clips 28 could also be manufactured from biologic or biologically reabsorbable materials that degrade or are absorbed over time as they become nonstructural. Such biologic snap-on augments 26 would also allow multiple augments to be snapped into place at multiple segments along the rim in cases of multidirectional instability, with other areas along the rim left un-augmented. This would help prevent impingement of the femoral component 14 in the un-augmented areas while helping optimize immediate postoperative range of movement and stability. Of course, it is within the scope of the invention for the snap-on semiannular augments 26 to include a combination of biologically acceptable, biologic, and biologically reabsorbable materials as desired.

In the exemplary embodiment utilizing the biologically reabsorbable snap-on augments 26, such augments 26 could be formulated to absorbed over a relatively short period (i.e., several weeks or months) and could also be formulated so as to be replaced by tissue (such as scar-tissue) that would provide for long-term hip stability and, hopefully, normal motion. Such formulations of biologic materials are well known by those of ordinary skill in the art.

As will be apparent to those of ordinary skill in the art there are many other biologic and/or biologically reabsorbable materials that can be used for the snap-on augments 26 or snap clips 28, and there are also new biologic materials being developed on a consistent basis, all of which fall within the scope of the invention.

Examples of biologic materials for use with the snap-on augments 26 and the male fasteners 28 include, without limitation, extra cellular matrices (ECMs). Examples of ECMs include, without limitation, porcine small intestine submucosa (SIS), xenogeneic small intestine submucosa (xSIS), urinary bladder submucosa (UBS), laminated intestinal submucosa, glutaraldehyde-treated bovine pericardium (GLBP). The biologic materials may be layered, molded, formed, braided, perforated, multilaminated, grafted or otherwise manipulated to achieve the desired properties and dimensions associated with the snap-on augments 26 and the male fasteners 28.

Examples of biologically reabsorbable materials for use with the snap-on augments 26 and the male fasteners 28 include, without limitation, MONOCRYL (poliglecaprone 25), PDS II (polydioxanone), surgical gut suture (SGS), gut, coated VICRYL (polyglactin 910, polyglactin 910 braided), human autograft tendon material, collagen fiber, POLYSORB, poly-L-lactic acid (PLLA), polylactic acid (PLA), polylactides (Pla), racemic form of polylactide (D,L-Pla), poly(L-lactide-co-D,L-lactide), 70/30 poly(L-lactide-co-D,L-lactide), polyglycolides (PGa), polyglycolic acid (PGA), polycaprolactone (PCL), polydioxanone (PDS), polyhydroxyacids, and resorbable plate material (see e.g. Orthopedics, October 2002, Vol. 25, No. 10/Supp.). The biologically reabsorbable materials may be layered, molded, formed, braided, perforated, multilaminated, grafted or otherwise manipulated to achieve the desired properties and dimensions associated with the snap-on augments 26 and the male fasteners 28. For example, the MONOCRYL (poliglecaprone 25), PDS II (polydioxanone), and resorbable plate materials may be block formed, while the surgical gut suture (SGS), gut, coated VICRYL (polyglactin 910), human autograft tendon material, collagen fiber, POLYSORB, poly-L-lactic acid (PLLA), polylactic acid (PLA), polyglycolic acid, and porcine small intestinal submucosa (SIS) material may be layered and formed. It is within the scope and spirit of the present invention that any of the above materials and techniques may be used individually, alternatively, or in conjunction to produce the snap-on augments 26 and the male fasteners 28.

It is also within the scope of the present invention to “load” (disburse, coat, impregnate, etc.) the biologic and/or biologically reabsorbable materials comprising the snap-on augments 26 and the male fasteners 28 with agents that could hasten or assist in tissue development, assist in clotting, and/or fight infection. Exemplary agents may include, for example, without limitation, concentrated platelets (SYMPHONY from Depuy Orthapedic) and gentamicin.

It is within the scope of the invention to incorporate growth stimulating factors in the above exemplary embodiments incorporating biologic or biologically reabsorbable materials. Examples of such growth stimulating factors include, without limitation, growth factor beta (GFB-β), basic fibroblast growth factor (bFGF), fibroblast growth factor (FGF), epidermal growth factor (EFG), transforming growth factor-β1 (TGF-β1), vascular endothelial growth factor (VEGF), connective tissue growth factor (CTGF), platelet-derived growth factor (PDGF), direct-mediated gene transfer, fibroblast-mediated gene transfer, myoblast-mediated gene transfer, TGF-β gene family, adenovirus-mediated gene transfer, recombinant adenovirus-induced tendon adhesion formation, BMP-12, bone morphogenetic protein-2 gene transfer, growth and differentiation factor-5 (GDF-5) and, insulin like growth factor (IFG). (See e.g. Koski et al., “Tissue-Engineered Ligament—Cells, Matrix, and Growth Factors”, July 2000 Tissue Engineering in Orthopedic Surgery, Volume 31, No. 3), (see e.g., Boyer, “Using Growth Factors to Enhance Tendon and Ligament Repair”, Orthopaedic Research Society Symposia, AAOS Annual Meeting New Orleans February 2003). Several of these growth factors have been proposed as possible mitogens in fibroblast growth.

It is also within the scope of the invention to incorporate connective tissue stem cells and progenitors with the biologic or biologically reabsorbable materials disclosed in the above embodiments. These connective tissue stem cells and progenitors may be incorporated into the snap-on augments 26 and the male fasteners 28 to provide a three dimensional framework for the creation of engineered tissue. Examples of such connective tissue stem cells and progenitors include, without limitation, fibroblastic colony-forming cells, fibroplast colony-forming units (CFU-F), bone marrow stromal cells, mesenchymal stem cells (MSC), and vascular pericytes. (See e.g. Meschler et al. “Connective Tissue Progenitors: Practical Concepts for Clinical Applications”, 2002 Clinical Orthopaedics and Related Research, No. 395, pp. 66-80).

It is also within the scope of the invention to incorporate hematopoietic stem cells and progenitors with the biologic or biologically reabsorbable materials disclosed in the above embodiments. These hematopoietic stem cells and progenitors may be incorporated into the snap-on augments 26 and the male fasteners 28 to provide any cell making up circulating blood and the immune system for, in an exemplary application, inhibiting infection after surgery.

Following from the above description and invention summaries, it should be apparent to those of ordinary skill in the art that, while the apparatuses and methods herein described constitute exemplary embodiments of the present invention, it is to be understood that the inventions contained herein are not limited to these precise embodiments and that changes may be made to them without departing from the scope of the inventions as defined by the claims. Additionally, it is to be understood that the invention is defined by the claims and it not intended that any limitations or elements describing the exemplary embodiments set forth herein are to be incorporated into the meanings of the claims unless such limitations or elements are explicitly listed in the claims. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. A prosthetic device for use with a hip replacement prosthesis that includes an acetabular cup assembly adapted to be fastened to a patient's pelvis and a femoral stem adapted to be fastened to the patient's femur, where the femoral stem includes a ball component at its proximal end received within the acetabular cup assembly to form a ball joint type coupling, the constraining device comprising:
a semiannular augment adapted to be mounted approximate to a rim of an acetabular cup assembly of a hip replacement prosthesis, wherein the augment assists in improving stability, at least temporarily, of a ball joint type coupling between the acetabular cup assembly and a femoral stem of the hip replacement prosthesis;
the semiannular augment being formed from an augment material selected from the group consisting of a biologic material, a biologically absorbable material, and a combination of biologic and biologically absorbable materials.

2. The prosthetic device of claim 1, further comprising at least one fastener for mounting the semiannular augment to the acetabular cup assembly, the fastener being formed from a fastener material selected from the group consisting of a biologic material, a biologically absorbable material, and a combination of biologic and biologically absorbable materials.

3. The prosthetic device of claim 2, wherein the fastener material includes at least one, or an equivalent, of:
a poly-L-lactic acid material; and
collagen.

4. The prosthetic device of claim 2, wherein the fastener comprises at least one of:
a screw;
a snap;
a clip;
a keyway;
a dowel; and
a rivet.

5. The prosthetic device of claim 1, wherein the augment material includes at least one, or an equivalent, of:
extra cellular matrices (ECMs);
poliglecaprone 25;
polydioxanone;
surgical gut suture (SGS);
gut;
polyglactin 910;
human autograft tendon material;
collagen fiber;
poly-L-lactic acid (PLLA);
polylactic acid (PLA);
polylactides (Pla);
racemic form of polylactide (D,L-Pla);
poly(L-lactide-co-D,L-lactide);
polyglycolides (PGa);
polyglycolic acid (PGA);
polycaprolactone (PCL);
polydioxanone (PDS);
polyhydroxyacids; and
resorbable plate material.

6. The prosthetic device of claim 5, wherein the extra cellular matrices (ECMs) include at least one of:
porcine small intestine submucosa (SIS);
xenogeneic small intestine submucosa (xSIS);
urinary bladder submucosa (UBS);
laminated intestinal submucosa; and
glutaraldehyde-treated bovine pericardium (GLBP).

7. The prosthetic device of claim 1, wherein a distal surface of the semiannular augment opposing a surface of the semiannular augment abutting the rim of the acetabular cup assembly is contoured to approximate the shape of a portion of the neck of the femoral component potentially coming into contact therewith.

8. The prosthetic device of claim 1, wherein the semiannular augment is positioned on an anterior/superior portion of the rim of the acetabular cup assembly.

9. The prosthetic device of claim 1, wherein:
the femoral component includes a ball at its proximal end for mating with the acetabular cup assembly to form a ball-joint coupling; and
the semiannular augment includes a contoured, radially inner surface to approximate an outer surface of the ball of the femoral component potentially coming into contact therewith.

10. The prosthetic device of claim 9, wherein the contoured, radially inner surface of the augment is substantially semi-spherically shaped.

11. The prosthetic device of claim 9, wherein the contoured, radially inner surface is substantially arcuate.

12. The prosthetic device of claim 1, wherein the semiannular augment is mounted to a proximal rim surface of the acetabular cup assembly.

13. The prosthetic device of claim 1, wherein the semiannular augment is mounted to a proximal rim surface of a cup-shaped bearing insert component of the acetabular cup assembly.

14. The prosthetic device of claim 2, wherein the semiannular augment includes at least one integrated fastener.

15. The prosthetic device of claim 14, wherein the integrated fastener includes a snap-on retention member enabling snap-on-type mounting of the semiannular augment to the acetabular cup assembly.

16. The prosthetic device of claim 1, wherein the augment material is supplemented with an agent to promote the formation of scar tissue.

17. The prosthetic device of claim 1, wherein the augment material is supplemented with a clotting agent.

18. The prosthetic device of claim 1, wherein the augment material is supplemented with an antibacterial agent.

19. The prosthetic device of claim 1, wherein the augment material is adapted to be substantially absorbed by a patient's body after implantation and to be substantially replaced by scar tissue.

20. The prosthetic device of claim 19, wherein the augment material is adapted to be substantially absorbed and replaced by scar tissue within approximately 6 months after implantation.

21. The prosthetic device of claim 1, wherein the semiannular augment extends less than 360 degrees about the femoral stem.

22. The prosthetic device of claim 1, wherein the semiannular augment extends 180 degrees or less about the femoral stem.

23. The prosthetic device of claim 1, wherein the semiannular augment extends 90 degrees or less about the femoral stem.

24. The prosthetic device of claim 1, wherein the semiannular augment extends 45 degrees or less about the femoral stem.

25. The prosthetic device of claim 1, wherein:
the femoral component includes a ball at its proximal end for mating with the acetabular cup assembly to form a ball-joint connection; and
the semiannular augment assists in constraining the ball within the acetabular cup assembly.

26. The prosthetic device of claim 1, wherein:
the femoral component includes a ball at its proximal end for mating with the acetabular cup assembly to form a ball-joint connection; and
the semiannular augment assists in restraining the ball within the acetabular cup assembly.

27. A hip prosthesis comprising:
an acetabular cup assembly adapted to be fastened to a patient's pelvis;
a femoral stem adapted to be fastened to the patient's femur, the femoral stem including a ball component at its proximal end received within the acetabular cup assembly to form a ball joint type coupling; and
a semiannular augment mounted to a distal end of the acetabular cup assembly, adjacent to the ball component, wherein the semiannular augment assists in stabilizing the ball joint type coupling between the acetabular cup assembly and the femoral stem;
the semiannular augment being formed from an augment material selected from the group consisting of a biologic material, a biologically absorbable material, and a combination of biologic and biologically absorbable materials.

28. The hip prosthesis of claim 27, further comprising at least one fastener mounting the semiannular augment to a distal rim surface of the acetabular cup assembly, wherein the fastener is formed from a fastener material selected from the group consisting of a biologic material, a biologically absorbable material, and a combination of biologic and biologically absorbable materials.

29. The hip prosthesis of claim 28, wherein the fastener material includes at least one, or an equivalent, of:
a poly-L-lactic acid material; and
collagen.

30. The hip prosthesis of claim 28, wherein the fastener comprises at least one of:
a screw;
a snap;
a clip;
a keyway;
a dowel; and
a rivet.

31. The hip prosthesis of claim 27, wherein the augment material includes at least one, or an equivalent, of:
extra cellular matrices (ECMs);
poliglecaprone 25;
polydioxanone;
surgical gut suture (SGS);
gut;
polyglactin 910;
human autograft tendon material;
collagen fiber;
poly-L-lactic acid (PLLA);
polylactic acid (PLA);
polylactides (Pla);
racemic form of polylactide (D,L-Pla);
poly(L-lactide-co-D,L-lactide);
polyglycolides (PGa);
polyglycolic acid (PGA);
polycaprolactone (PCL);
polydioxanone (PDS);
polyhydroxyacids; and
resorbable plate material.

32. The hip prosthesis of claim 31, wherein the extra cellular matrices (ECMs) include at least one of:
porcine small intestine submucosa (SIS);
xenogeneic small intestine submucosa (xSIS);
urinary bladder submucosa (UBS);
laminated intestinal submucosa; and
glutaraldehyde-treated bovine pericardium (GLBP).

33. The hip prosthesis of claim 27, wherein a distal surface of the semiannular augment is contoured to approximate the shape of a portion of the neck of the femoral component.

34. The hip prosthesis of claim 27, wherein the semiannular augment is positioned on the anterior/superior portion of the acetabular cup assembly.

35. The hip prosthesis of claim 27, wherein the semiannular augment includes a contoured, radially inner surface to approximate an outer surface of the ball of the femoral stem.

36. The hip prosthesis of claim 27, wherein the contoured, radially inner surface of the semiannular augment is substantially semi-spherically shaped.

37. The hip prosthesis of claim 27, wherein the augment material is supplemented with an agent to promote the formation of scar tissue.

38. The hip prosthesis of claim 27, wherein the augment material is supplemented with a clotting agent.

39. The hip prosthesis of claim 27, wherein the augment material is supplemented with an antibacterial agent.

40. The hip prosthesis of claim 27, wherein the augment material is adapted to be substantially absorbed by a patient's body after implantation and to be substantially replaced by scar tissue.

41. The hip prosthesis of claim 40, wherein the augment material is adapted to be substantially absorbed and replaced by scar tissue within approximately 6 months after implantation.

42. The hip prosthesis of claim 27, wherein the semiannular augment extends less than 360 degrees about the femoral stem.

43. The hip prosthesis of claim 27, wherein the semiannular augment extends 180 degrees or less about the femoral stem.

44. The hip prosthesis of claim 27, wherein the semiannular augment extends 90 degrees or less about the femoral stem.

45. The hip prosthesis of claim 27, wherein the semiannular augment extends 45 degrees or less about the femoral stem.

46. The hip prosthesis of claim 27, wherein the semiannular augment assists in constraining the ball within the acetabular cup assembly.

47. The hip prosthesis of claim 27, wherein the semiannular augment assists in restraining the ball within the acetabular cup assembly.

48. The hip prosthesis of claim 27, comprising a plurality of the semiannular augments mounted to the distal end of the acetabular cup assembly, adjacent to the ball component.

49. The hip prosthesis of claim 48, wherein a first one of the semiannular augment restrains the ball within the acetabular cup assembly and a second one of the semiannular augments is adapted to abut the femoral stem when the femoral stem pivots to a predetermined angle.

50. The hip prosthesis of claim 49, wherein the second semiannular augment is diametrically opposed to the first semiannular augment about the acetabular cup assembly.

51. The hip prosthesis of claim 27, wherein the acetabular cup assembly includes a cup-shaped bearing insert receiving the ball component of the femoral stem and the semiannular augment is mounted to a rim of the bearing insert.

52. The hip prosthesis of claim 51, wherein a first one of the semiannular augment restrains the ball within the acetabular cup assembly and a second one of the semiannular augments is adapted to abut the femoral stem when the femoral stem pivots to a predetermined angle.

53. The hip prosthesis of claim 52, wherein the second semiannular augment is diametrically opposed to the first semiannular augment about the acetabular cup assembly.

54. A prosthetic constraining kit for implantation in proximity to a hip joint, comprising a plurality of constraining augments being adapted to be individually fastened on at least one of an acetabular prosthesis and about an acetabular cavity within a hip bone and circumferentially positionable about a femoral member taken from a group consisting of a femur and a femoral prosthesis, where the constraining augments at least partially define a central aperture allowing the femoral member to extend therethrough and allowing a range of angular motion of the femoral member while inhibiting a femoral head of the femoral member from completely passing distally through the central aperture.

55. The prosthetic constraining kit of claims 54, wherein the constraining augments comprise at least one of a biologic material, a biologically absorbable material, and a combination of biologic and biologically absorbable materials.

56. The prosthetic constraining kit of claim 54, wherein the constraining augments are circumferentially positioned to define a ring.

57. The prosthetic constraining kit of claim 54, wherein at least two of the augments are spaced apart to define a discontinuous ring.

58. The prosthetic constraining kit of claim 57, wherein the discontinuous ring includes more than one discontinuity.

59. The prosthetic constraining kit of claim 54, wherein each constraining augment includes at least one fastener for mounting the augment to the distal rim surface of the acetabular cup assembly, wherein the fastener is formed from a fastener material selected from the group consisting of a biologic material, a biologically absorbable material, and a combination of biologic and biologically absorbable materials.

60. The prosthetic constraining kit of claim 59, wherein the fastener material includes at least one, or an equivalent, of:
a poly-L-lactic acid material; and
collagen.

61. The prosthetic constraining kit of claim 59, wherein the fastener comprises at least one of:
a screw;
a snap;
a clip;
a keyway;
a dowel; and
a rivet.

62. The prosthetic constraining kit of claim 55, wherein the material of the constraining augments includes at least one, or an equivalent, of:
extra cellular matrices (ECMs);
poliglecaprone 25;
polydioxanone;
surgical gut suture (SGS);
gut;
polyglactin 910;
human autograft tendon material;
collagen fiber;
poly-L-lactic acid (PLLA);
polylactic acid (PLA);
polylactides (Pla);
racemic form of polylactide (D,L-Pla);
poly(L-lactide-co-D,L-lactide);
polyglycolides (PGa);
polyglycolic acid (PGA);
polycaprolactone (PCL);
polydioxanone (PDS);
polyhydroxyacids; and
resorbable plate material.

63. The prosthetic constraining kit of claim 62, wherein the extra cellular matrices (ECMs) include at least one of:
porcine small intestine submucosa (SIS);
xenogeneic small intestine submucosa (xSIS);
urinary bladder submucosa (UBS);
laminated intestinal submucosa; and
glutaraldehyde-treated bovine pericardium (GLBP).

64. The prosthetic constraining kit of claim 54, wherein a distal surface of at least one constraining augment is contoured to approximate the shape of a portion of the neck of the femoral member.

65. The prosthetic constraining kit of claim 54, wherein at least one constraining augment is positioned on the anterior/superior portion of the acetabular cup assembly.

66. The prosthetic constraining kit of claim 54, wherein at least one of the constraining augments has an inner radial surface that is substantially semi-spherically shaped to complement the shape of the femoral head of the femoral member.

67. The prosthetic constraining kit of claim 59, wherein the at least one fastener is an integrated fastener including snap-on retention members enabling snap-on mounting to the acetabular cup assembly.

68. The prosthetic constraining kit of claim 55, wherein the material of at least one of the constraining augments is supplemented with an agent to promote the formation of scar tissue.

69. The prosthetic constraining kit of claim 55, wherein the material of at least one of the constraining augments is supplemented with a clotting agent.

70. The prosthetic constraining kit of claim 55, wherein the material of at least one of the constraining augments is supplemented with an antibacterial agent.

71. The prosthetic constraining kit of claim 54, wherein a distal surface of at least one of the constraining augments further includes an elevated portion for reducing angular movement of the femoral member in the radial direction of the elevated portion.

72. The prosthetic constraining kit of claim 71, wherein the elevated portion is located in a radially outer region of the constraining augment when mounted to one of the acetabular prosthesis and an area surrounding the acetabular cavity within the hip bone.

73. A constraining device for, at least temporarily, promoting engagement of a prosthetic femoral stem component with a prosthetic acetabular component of a prosthetic hip assembly, the constraining device comprising a semiannular segment of material selected from the group consisting of a biologic material, a biologically absorbable material, and a combination of biologic and biologically absorbable materials.

74. The constraining device of claim 73, wherein the semiannular segment of material includes at least one, or an equivalent, of:
extra cellular matrices (ECMs);
poliglecaprone 25;
polydioxanone;
surgical gut suture (SGS);
gut;
polyglactin 910;
human autograft tendon material;
collagen fiber;
poly-L-lactic acid (PLLA);
polylactic acid (PLA);
polylactides (Pla);
racemic form of polylactide (D,L-Pla);
poly(L-lactide-co-D,L-lactide);
polyglycolides (PGa);
polyglycolic acid (PGA);
polycaprolactone (PCL);
polydioxanone (PDS);
polyhydroxyacids; and
resorbable plate material.

75. The constraining device of claim 74, wherein the extra cellular matrices (ECMs) includes at least one of:
porcine small intestine submucosa (SIS);
xenogeneic small intestine submucosa (xSIS);
urinary bladder submucosa (UBS);
laminated intestinal submucosa; and
glutaraldehyde-treated bovine pericardium (GLBP).

76. The constraining device of claim 73, wherein the semiannular segment of material is molded in the form of a constraining ring adapted to be segmented and mounted to a rim of an acetabular cup assembly of the acetabular prosthesis component.

77. The constraining device of claim 73, wherein the semiannular segment of material is adapted to be substantially absorbed by a patient's body after implantation and to be substantially replaced by scar tissue.

78. The constraining device of claim 77, wherein the semiannular segment of material is adapted to be substantially absorbed and replaced by scar tissue within approximately 6 months after implantation.

79. The constraining device of claim 73, wherein the semiannular segment of material is supplemented with an agent to promote the formation of scar tissue.

80. The constraining device of claim 73, wherein the semiannular segment of material is supplemented with a clotting agent.

81. The constraining device of claim 73, wherein the semiannular segment of material is supplemented with an antibacterial agent.

82. A restraining device for, at least temporarily, promoting engagement between at least two of a first prosthetic joint component, a second prosthetic joint component, a first bone component, and a second bone component, wherein the restraining device is comprised of a restraining material including at least one of a biologic material, a biologically absorbable material, and a combination of biologic and biologically absorbable materials, wherein the restraining device does not circumscribe at least one of the first prosthetic joint component, the second prosthetic joint component, the first bone component, and the second bone component.

83. The restraining device of claim 82, wherein the restraining material includes at least one, or an equivalent, of:
extra cellular matrices (ECMs);
poliglecaprone 25;
polydioxanone;
surgical gut suture (SGS);
gut;
polyglactin 910;
human autograft tendon material;
collagen fiber;
poly-L-lactic acid (PLLA);
polylactic acid (PLA);
polylactides (Pla);
racemic form of polylactide (D,L-Pla);
poly(L-lactide-co-D,L-lactide);
polyglycolides (PGa);
polyglycolic acid (PGA);
polycaprolactone (PCL);
polydioxanone (PDS);
polyhydroxyacids; and
resorbable plate material.

84. The restraining device of claim 83, wherein the extra cellular matrices (ECMs) includes at least one of:
porcine small intestine submucosa (SIS);
xenogeneic small intestine submucosa (xSIS);
urinary bladder submucosa (UBS);
laminated intestinal submucosa; and
glutaraldehyde-treated bovine pericardium (GLBP).

85. The restraining device of claim 82, wherein the restraining device is adapted to be mounted to at least one of the first prosthetic joint component, the second prosthetic joint component, the first bone component, and the second bone component.

86. The restraining device of claim 82, wherein the restraining device is adapted to be substantially absorbed by a patient's body after implantation and to be substantially replaced by scar tissue.

87. The restraining device of claim 82, wherein the restraining material is adapted to be substantially absorbed and replaced by scar tissue within approximately 6 months after implantation.

88. The restraining device of claim 82, wherein the restraining material is supplemented with an agent to promote the formation of scar tissue.

89. The restraining device of claim 82, wherein the restraining material is supplemented with a clotting agent.

90. The restraining device of claim 82, wherein the restraining material is supplemented with an antibacterial agent.

91. A method for providing at least temporary stability to a prosthetic hip joint that includes an acetabular cup assembly bonded to a patient's pelvis and a femoral stem bonded to the patient's femur, where the femoral stem includes a ball component at its proximal end received within the acetabular cup assembly to form a ball joint coupling, the method comprising the step of:
mounting a stability enhancement augment to the prosthetic hip joint to improve, at least temporarily, the stability of the prosthetic hip joint, wherein the stability enhancement augment is formed from an augment material selected from the group consisting of a biologic material, a biologically absorbable material, and a combination of biologic and biologically absorbable materials.

92. The method of claim 91, wherein:
the stability enhancement augment maximally exhibits a semiannular shape covering less than 360 degrees and partially defines an aperture; and
the mounting step includes the step of mounting the stability enhancement augment about a rim of the acetabular cup assembly where the femoral stem passes through the aperture.

93. The method of claim 91, wherein the mounting step includes the step of fastening the stability enhancement augment to the rim of the acetabular cup assembly with at least one fastener formed from a fastener material comprising at least one of a biologic material, a biologically absorbable material, and a combination of biologic and biologically absorbable materials.

94. The method of claim 93, wherein the fastener comprises at least one of:
a screw;
a snap;
a clip;
a keyway;
a dowel; and
a rivet.

95. The method of claim 91, wherein the augment material is loaded with at least one of an agent to promote formation of scar tissue, a clotting agent, and an antibacterial agent.

96. The method of claim 91, wherein the augment material is adapted to be substantially absorbed by a patient's body after the mounting step and to be substantially replaced by scar tissue.

97. The method of claim 91, wherein the stability enhancement augment is in the form of a paste material and the mounting step includes the step of applying the paste material to at least a portion of the patient's prosthetic hip joint.

98. The method of claim 97, wherein the augment material is loaded with at least one of an agent to promote formation of scar tissue, a clotting agent, and an antibacterial agent.

99. The method of claim 98, wherein the augment material is adapted to be substantially absorbed by a patient's body after the mounting step and to be substantially replaced by scar tissue.

100. The method of claim 99, wherein the augment material is adapted to be substantially absorbed and replaced by scar tissue within approximately 6 months after implantation.

101. A method for providing at least temporary stability to a prosthetic hip joint which includes an acetabular cup assembly bonded to a patient's pelvis and a femoral stem bonded to the patient's femur, where the femoral stem includes a ball component at its proximal end received within the acetabular cup assembly to form a ball joint type coupling, the method comprising the step of:
mounting a plurality of individual constraining augments on an acetabular prosthesis, where the individual constraining augments at least partially define a central aperture for allowing a femoral component to extend therethrough while inhibiting a femoral head of the femoral component from completely passing distally through the central aperture.

102. The method of claim 101, wherein the mounting step includes selectively manipulating the contour of the constraining augments to customize the shape of the augments.

103. The method of claim 101, wherein the mounting step includes the step of selectively positioning the constraining augments circumferentially about the acetabular prosthesis.

104. The method of claim 101, wherein the mounting step includes excavating the acetabular prosthesis.

105. The method of claim 101, wherein the mounting step includes excavating the constraining augments.

106. The method of claim 101, wherein the mounting step includes inserting at least one of a fastener coupled to the acetabular prosthesis, a fastener independent of the acetabular prosthesis, and a glue into a cavity within the acetabular prosthesis.

107. The method of claim 101, wherein the constraining augments are adapted to be substantially absorbed by a patient's body after implantation and to be substantially replaced by scar tissue.

108. The method of claim 86, wherein the constraining augments are adapted to be substantially absorbed and replaced by scar tissue within approximately 6 months after implantation.
